(19) United States
(12) Patent Application Publication
(10) Pub. No.: US 2006/0199211 A1
(43) Pub. Date: Sep. 7, 2006

(54) Fluorous-based microarrays

(76) Inventors: Nicola Lucia Pohl, Ames, IA (US); Kwang-Seuk Ko, Ames, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 11/368,045

(22) Filed: Mar. 3, 2006

Related U.S. Application Data
(60) Provisional application No. 60/658,347, filed on Mar. 3, 2005.
(60) Provisional application No. 60/710,327, filed on Aug. 22, 2005.

(51) Int. Cl. C12Q 1/68; G01N 33/53; C12M 1/34

(52) U.S. Cl. 435/6; 435/7.1; 435/287.2

(57) ABSTRACT

A novel means of arraying fluorous tagged probes is disclosed. The method involves fluorous-tagging probes and attaching the same to a substrate having a fluorous surface. Target molecules are then identified and analyzed following attachment to the microarray.

### CROSS-REFERENCE TO RELATED APPLICATIONS

This application claims priority under 35 U.S.C. § 119 of a provisional application Ser. No. 60/658,347 filed Mar. 3, 2005, and from provisional application Ser. No. 60/710,327 filed Aug. 22, 2005, which applications are hereby incorporated by reference in their entirety.

### GRANT REFERENCE CLAUSE

This invention was funded in part by grant NSF Contract No. MCB0349139. The government may have certain rights in this invention.

### BACKGROUND OF THE INVENTION

A microarray is an array of spots of biological or chemical samples (probes) immobilized at predefined positions on a substrate. Each spot contains a number of molecules of a single biological or chemical material. To analyze the array, the microarray is flooded with a fluid containing one or more biological or chemical samples, elements of which typically interact with one or more complementary probes on the microarray. The molecular strands in the target hybridize with complementary strands in the probe microarray. The hybridized microarray is inspected by a microarray reader, which detects the presence of the target molecules. Since the probes are placed in predetermined and thus known positions in the microarray, the presence and quantity of target sequences in the fluid are identified by the position at which fluorescence or radiation is detected and the strength of the fluorescence or radiation.

The success of DNA and protein microarrays in biological screening with a minimal amount of material has lead to related chip-based approaches to probe carbohydrate-protein interactions. Generally, carbohydrate microarrays are prepared by the site-specific covalent attachment of chemically modified sugars to an appropriately derivatized surface. For example, maleimide- or hydrazide-linked carbohydrates can react with thiol- or epoxide-derivatized glass slides, and cyclopentadiene-containing carbohydrates can be immobilized on a benzoquinone-coated gold surface by Diels-Alder reactions. Unfortunately, such covalent attachment strategies require the tedious optimization of immobilization conditions such as pH, time, and temperature. The noncovalent immobilization of carbohydrates has been carried out using nitrocellulose-coated glass slides. However, this method requires larger oligosaccharides and allows no control over sugar orientation on the surface.

Recently, carbohydrate microarrays have been fabricated based on another type of noncovalent interaction. These experiments were the first demonstration that non-covalent fluorous-fluorous interactions could be used not only for simplified purification but also for surface patterning of compounds. In fact, a single C₈F₁₇ fluorous tail is sufficient to pattern carbohydrates in arrays on a fluorinated solid support and to retain the compounds during routine screening against carbohydrate-binding proteins such as plant lectins. These initial experiments, however, used only simple monosaccharides.

Microarrays are a significant advance both because they may contain a very large number of molecules and because of their small size. Microarrays are therefore useful when one wants to survey a large number of molecules quickly or when the sample to be studied is small. For instance, microarrays may be used to assay gene expression within a single sample or to compare gene expression in two different cell types or tissue samples, such as in healthy and diseased tissue. Because a microarray can be used to examine the expression of hundreds or thousands of molecules at once, it promises to revolutionize the way these molecules are examined and studied. As more information accumulates, scientists will be able to use microarrays to ask increasingly complex questions and perform more intricate experiments.

It is a primary objective of the present invention to provide a simple means of forming microarrays that may be used in biological screening methods.

It is a further objective to develop improved techniques for screening sugars, proteins, and combinations of the same.

It is still a further objective of the present invention to provide a method of biologically screening molecules using microarrays.

These and other objectives will become clear from the following detailed description of the invention.

### SUMMARY OF THE INVENTION

The present invention describes novel microarrays and multi-well plate arrays for synthesizing fluorous-tagged probes, and methods of forming the same. The microarrays include a slide, well, or other substrate having a fluorous surface capable of noncovalently attaching fluorous-tagged probes. Once attached, target molecules bind to the probes and can thereafter be detected using conventional means. The present invention is advantageous over previous microarrays in allowing direct array formation using non-covalent fluorous-based interactions.

### DETAILED DESCRIPTION OF THE INVENTION

The present invention provides relatively simpler methods of forming microarrays based on noncovalent fluorous-based interactions. In contrast to the production of microarrays from compounds made on solid-phase that require multiple solution-phase and deprotection and derivation steps, the present invention allows direct array formation, a property that oligomer synthesis methods on solid phase or with lipid or polyethylene glycol tags do not share.

The microarray substrate is prepared having fluorous surfaces to noncovalently attach fluorous tagged compounds. The substrate of the invention can be made of various materials. The substrate is required to be capable of immobilizing the particular probes used, or the substrate must be capable of modification (for example, by coating) so that it is capable of such immobilization. Preferred materials for the substrate of the present invention include silica, glass, metals, plastics, and polymers. Any microarray substrate having fluorous surfaces is suitable for use in the invention, including Teflon® coated slides (such as those manufactured by Precision Lab Products) or any other Teflon® coated surfaces. As used herein, the term “fluorous surface” is defined as sufficient fluorocarbons for water to form beads on the surface of the substrate and/or a surface having sufficient fluorocarbons to bind the fluorous tags chosen by the user.

In the alternative, substrates having fluorinated surfaces can also be readily manufactured. Preferably, the slides or other substrate are first cleaned using ethanol or other conventional means. The substrate is then immersed in a fluorous solution to coat the substrate, which is then dried and washed. In a preferred manufacturing method, the substrate is immersed at least once in a solution of Rf8-reagent, such as Rf8-ethyl-SiCl₃. In a most preferred method, the slides are immersed multiple times, with three times being most preferred, with periods of drying for a few minutes each time in between.

In an alternative but less preferred method of the invention, commercially available fluorous amine can be reacted with maleic anhydride on the surface of the substrate. Alternatively, the corresponding fluorous thiol can be reacted with commercially available maleimide activated plates to produce glass slides or wells with fluorous surfaces, as shown below. As noted above, when properly coated, water will bead up on the fluorous surfaces.

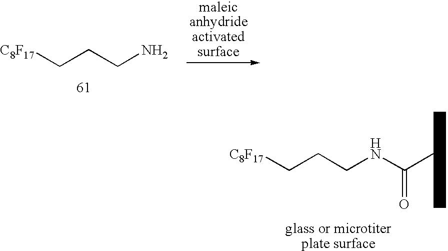

Once the microarray substrate is prepared, fluorous-tagged molecules (probes) are spotted on the coated substrate. Fluorous tags are perfluoroalkyl modified versions of traditional protecting groups that are soluble in most organic solvents, such as DMF, THF, CH₂Cl₂ etc. The tag needs to survive the necessary sequential reaction conditions and also be removed if desired. Suitable fluorous tags for use in the present invention include, but are not limited to, F-Boc-ON, F-thiol, F-Cbz-OSu, F-silanes, fluorous benzyl alcohol, F-Fmoc-Osu, F-PMB-OH, FluoMarg, fluorous trityl chlorides, fluorous t-butanols (i.e. C₄F₉, C₆F₁₃, etc.), 4-[3-(perfluorooctyl)-propyl-1-oxy]-thiophenol, fluorous-ter-butyldiphenylsilyl, fluorous-para-methyoxybenzyl, fluorous-fluorenylmethoxycarbonyl, OTf (F-TBDPS trifuloromethanesulfonate), fluorous levulinate (F-Lev), fluorous allyl groups (F-Allyl), and fluorous trityl alcohols. Fluorous tags are well known in the art and are commercially available from such companies as Fluorous Technologies Incorporated (FTI). The fluorous tags preferably have a general formula of R—CₓF₍₂ₓ₊₁₎, whereby R comprises one or alkyl groups and x is an integer. C₈F₁₇ tags are preferred for reasons of convenience, i.e. readily accessible commercial availability.

The present invention also includes unique fluorous tags, fluorous levulinate (F-Lev) and fluorous allyl (F-Allyl). F-Lev may be synthesized by formation of the Grignard reagent from 2-(perfluorohexyl)ethyl iodide followed by addition of succinic anhydride.

An allyl group works well with standard trichloroacetimidate coupling conditions and deprotection conditions. Reaction of cis-1,4-butenediol with 1H,1H,2H,2H-perfluorodecyl iodide using mild base conditions (Manzoni 2004) produces an alcohol with the requisite alkene spacer for use in glycosylations. The present inventors developed a means of synthesizing a modified version of this tag on a larger scale. An additional methylene spacer between the leaving group and the electron-withdrawing fluoroalkane was added to reduce the acidity of the hydrogen beta to the leaving group. To this end, commercially available 3-(perfluorooctyl)propanol was mixed with methanesulfonyl chloride to provide mesylate derivative quantitatively. This electrophile was then reacted with cis-1,4-butenediol to produce fluorous-tagged alcohol in 70% yield for use in subsequent glycosylation reactions.

The procedure for tagging molecules having protecting groups is well known in the art. The conditions will vary depending upon the tag(s) chosen, substrate used, etc. An exemplary and well known reference in this respect is T. W. Green, P. G. M. Wuts, Protective Groups in Organic Synthesis, Wiley-Interscience, New York, 1999, the contents of which are specifically incorporated herein by reference. This book provides detailed information to persons skilled in the art regarding the tagging conditions/procedures to use depending on the protecting group selected.

The tagged molecules or “probes”, are immobilized on the substrate in accordance with the invention by noncovalent attachment. The probes are specifically bound to a target material to be detected, and may be, but are not limited to, proteins or fragments of proteins, nucleotides (RNA, DNA, etc.), or carbohydrates (i.e. polysaccharides, oligosaccharides, or monosaccharides), molecular complexes (e.g., nucleic acid hybrids, protein complexes, cell components), reactive complexes (e.g., complexes undergoing chemical or enzymatic reactions), lipids and fatty acids, steroids, drugs, cells, tissue, and other small molecules (i.e. having a molecular weight of about or less than 10,000 Daltons). More particularly, the probes may be DNAs, RNAs, antibodies, antigens, ligands, substrates, or inhibitors. The set of probes chosen depends on the use of the apparatus. For example, if the apparatus uses polynucleotides as probes, if one is performing sequence analysis, one would prefer a complete or nearly complete set of n-mers; the use of such sets is more fully described in U.S. Pat. Nos. 5,700,637 and 6,054,270, which are hereby incorporated herein by reference in their entirety. On the other hand, if a device is to be used to analyze mutations or polymorphisms in a gene or set of genes, polynucleotides representing a complete or chosen set of mutations, such as substitution, deletion, and insertion mutations, for sections of the particular gene or genes of interest may be preferred. These examples are merely illustrative of the various custom sets of probes that might be selected for a particular apparatus and focus on polynucleotides because these are the types of probes now most commonly in use; it is to be understood that other types of probes and other sets of polynucleotides will be readily apparent to the skilled worker in the field.

The samples being deposited on the microarray substrate using the technology disclosed herein can take or be carried by any physical form that can be transported by a robotic pin or through a capillary. These include but not limited to fluid, gel, paste, bead, powder and particles suspended in liquid. Established robotic spotting techniques use a specially designed mechanical robot, which produces a probe spot on the microarray by dipping a pin head into a fluid containing an off-line synthesized DNA or other molecule and then spotting it onto the slide at a predetermined position. Washing and drying of the pins are required prior to the spotting of a different probe in the microarray. In current designs of such robotic systems, the spotting pin, and/or the stage carrying the microarray substrates move along the XYZ axes in coordination to deposit samples at controlled positions of the substrates. In addition to the established robotic spotting technologies, there are a number of microarray fabrication techniques that are being developed. These include the inkjet technology and capillary spotting.

The fluorous-tagged molecules are preferably dissolved in an appropriate solvent to form a solution, then spotted on the fluorinated substrate using the means described above. The substrate is then dried in a humidifying chamber or by other conventional drying means. The sample spot sizes in microarrays are typically less than 200 microns in diameter, and each array usually contains thousands of spots.

Once the probes are attached to the substrate, the sample containing the target material is contacted with the probes, typically by simply pouring the sample over the tagged substrate. Generally, the sample of a liquid phase contacts with the probes in a condition appropriate to induce the reaction between the target material and the probes. For example, in the case of detecting a carbohydrate target material, probes and target carbohydrates are incubated in a condition of optimal temperature and salt concentration that can induce the binding of the carbohydrate and carbohydrate binding proteins.

When specific reaction between the target material and the probes is completed, unreacted reactants can removed by washing or other conventional means.

To generate data from microarray assays some signal is detected that signifies the presence of, or absence of, the sequence of, or the quantity of the assayed compound or event. Detection of the reaction between the target material and the probes may be carried out by various methods, such as confocal fluorescent scanner, low luminescence detector, isotope imager, etc. A method of detecting an optical, electrical, or color signal may be used. According to an optical detection method, the target material is generally labeled with an optically detectable element. The reaction results between the target material and the probes can be detected by measuring light emitted from a reaction product of the target material and the probes by irradiation of excitation light. The invention is also intended to encompass technology and detection methods yet to be developed.

Fluorous-based microarray methods allow the facile formation of a range of carbohydrate chips for the plant and other sciences using synthetic carbohydrates produced with the aid of fluorous-tagged synthesis. This approach is especially valuable for the production of arrays containing compounds, such as glycosaminoglycan fragments, that contain nucleophiles that complicate current defined covalent attachment strategies.

The following examples are offered to illustrate but not limit the invention. Thus, they are presented with the understanding that various modifications may be made and still be within the spirit of the invention.

### EXAMPLE 1

### Tetrasaccharide Synthesis and Fluorous Coating of Substrate Surface in Preparation for Microarray Formation

As shown below, coupling between 1 and 2 was conducted using a catalytic amount of trimethylsilyl triflate (TMSOTf) or triflic acid as a promoter. Even if the reaction is incomplete, the desired disaccharide 3 can be separated from the rest of the materials by automated fluorous flash chromatography under optimized conditions. The acetate (Ac) group in 3 was then removed by sodium methoxide to give 4 that was purified by reverse phase SPE, or fluorous flash chromatography. With disaccharide 4 as a glycosyl acceptor, the same glycosylation-deprotection reactions is repeated two more times to give tetrasaccharide 5.

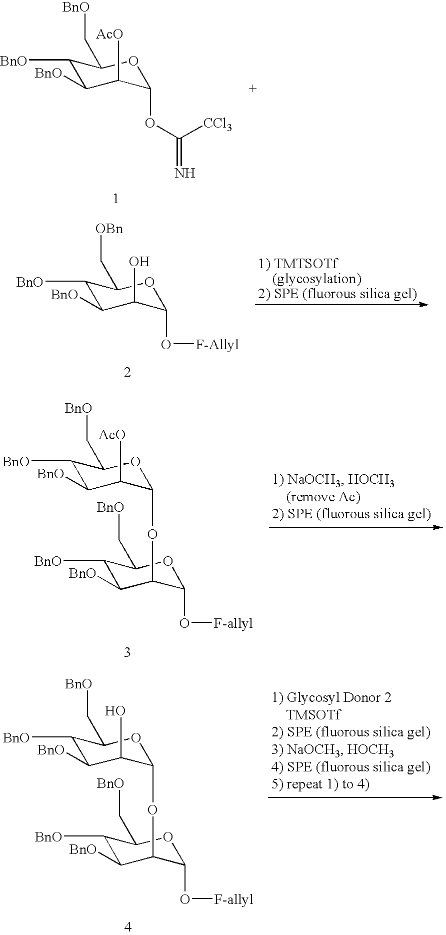

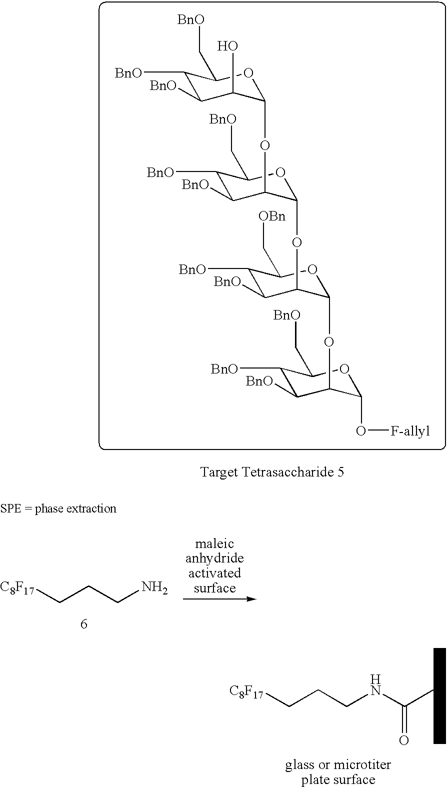

### EXAMPLE 2

### Synthesis and Testing of Microarrays

Tetrasaccharide 5, as well as the corresponding mono-, di-, and trisaccharide intermediates, were deprotected by hydrogenation with Pd/C. These compounds were taken up in aqueous methanolic solutions and drops deposited in the fluorous-phase 96-well microtiter plates. The methanol was then removed by slow evaporation to promote alignment of the fluorous-tagged compounds with the carbohydrate portion on the surface as reported for lipid interactions with hydrophobic wells (Fazio 2002). The commercially available lectin conconavalin A, which is known to bind to mannose (for example see: Weatherman, 1996), was then added to the wells. The hydrogenated allyl linker was used as an initial control compound. After rinsing of the solution with aqueous buffer, the amount of protein still bound will be measured by a Bradford assay (Bradford, 1976). The lectin bound to the wells containing the mannose sugars. These experiments serve to establish experimental parameters for forming compound arrays using fluorous tags as a foundation for Phase II work in making oligosaccharide arrays.

### EXAMPLE 3

### Preparation of Fluorous-Based Carbohydrate Microarrays

General Materials and Methods

Reaction solvents were distilled from calcium hydride for dichloromethane and from sodium metal and benzophenone for diethyl ether. Amberlyst 15 ion-exchange resin was washed repeatedly with methanol before use. All other commercial reagents and solvents were used as received without further purification. The reactions were monitored and the R_{f} values determined using analytical thin layer chromatography (tlc) with 0.25 mm EM Science silica gel plates (60F-254). The developed tlc plates were visualized by immersion in p-anisaldehyde solution followed by heating on a hot plate. Silica gel flash chromatography was performed with Selecto Scientific silica gel, 32-63 mm particle size. Fluorous phase chromatography was performed using fluorous solid-phase extraction cartridges containing silica gel bonded with perfluorooctylethylsilyl chains (FluorousTechnologies, Inc.; Pittsburgh, Pa.). All other fluorous reagents were also obtained from Fluorous Technologies, Inc. All moisture-sensitive reactions were performed in flame- or oven-dried glassware under a nitrogen atmosphere. Bath temperatures were used to record the reaction temperature in all cases run without microwave irradiation. All reactions were stirred magnetically at ambient temperature unless otherwise indicated. Microwave heating was carried out with a CEM-Discover continuous wave microwave. ¹H NMR and ¹³C NMR spectra were obtained with a Bruker DRX400 at 400 MHz and 162 MHz respectively. ¹⁷F spectrum was obtained with a Varian VXR400 at 376 MHZ. ¹H NMR spectra were reported in parts per million (δ) relative to CDCl₃ (7.27 ppm) as an internal reference. ¹³C NMR spectra were reported in parts per million (δ) relative to CDCl₃ (77.23 ppm) or CD₃OD (49.15 ppm). ¹⁹F NMR spectra were reported in parts per million (δ) relative to hexafluorobenzene C₆F₆ (0.00 ppm).

Microarray Preparation and Screening

Cleaning of glass slide. Glass slides were cleaned by immersion for 2 h in 60% ethanol containing NaOH (10 g/100 mL). The slides were washed twice in distilled water and then submerged in 95% ethanol.

Formation of fluorous-derivatized glass slides. Cleaned slides were immersed three times in a solution of Rf8-ethyl-SiCl₃ (3% solution 100 mL in methanol) and then dried for a few minutes; the process was repeated two more times. The slides were then washed twice by immersion in MilliQ-filtered water and then dried in a dessicator at room temperature.

Formation of carbohydrate microarrays. Fluorous-tagged carbohydrate compounds were dissolved in 60% methanol in water and spotted on the fluorinated glass slide using a robotic spotter (Cartesian PixSys 5500 Arrayer, Cartesian Technologies, Inc., Irvine, Calif.) at 30% humidity. The glass slide was dried in a humidifying chamber at 30% humidity for 2 h.

Detection of protein-carbohydrate binding. For Con A, FITC-labeled Con A in HEPES buffer (pH=7.5, 10 mM) 1 mM CaCl₂, 1 mM MnCl₂, 100 mM NaCl, 1% BSA (w/v) or without BSA, and for ECA, FITC-labeled ECA in PBS buffer (pH=6.8), 1.0% TWEEN 20, ECA (25 μg mL-1; EY Laboratories, Inc., San Mateo, Calif.) were used in the detection of protein-carbohydrate interactions. For protein incubation, 0.5 mL of protein solution was applied to the printed glass slide. The arrays was incubated by using a PC500 Cover Well incubation chamber (Grace Biolabs, Bend, Oreg.) and gently shaken every 5 min for 30 min. The slides were then washed three times with the incubation buffer followed by three washes with distilled water. The slides were subsequently dried for 30 min in a dark humidity chamber. The glass slide was scanned using a General Scanning ScanArray 5000 set at 488 nm.
Synthetic Procedures

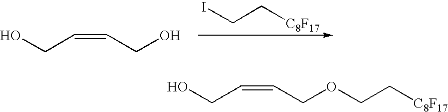

### Cis-1H,1H,2H,2H-perfluorodecyloxybutenyl alcohol

Cis-1,4-butenediol (140 mg, 1.59 mmol) was dissolved with DMF (20 mL). TBAB (153 mg, 48.0 mmol) and 1H,1H,2H,2H-perfluorodecyliodide (1.8 g, 3.18 mmol) were added to the solution. The reaction mixture was heated at 50° C. for 10 min; KOH (267 mg, 4.77 mmol) then was added into the mixture. The reaction mixture was heated at 70° C. for 30 min. The reaction mixture was poured into ice water. The organic layer was extracted with diethyl ether (100 mL×2) and dried over MgSO₄. The mixture was filtered and the solvent removed under reduced pressure. The crude product was purified by solid-phase extraction using a fluorous solid-phase extraction cartridge. The crude product was dissolved in DMF (0.5 mL) to load onto the fluorous solid-phase extraction cartridge. The crude product was absorbed onto the column and then 80% MeOH/water (10 mL) was used to wash the nonfluorous compounds through the column. The desired product was obtained by elution with 100% MeOH to yield, upon solvent removal, the alcohol as a colorless oil (1.12 g,

2.1 mmol, 67%).

R_{f}: 0.46 (ethyl acetate/hexane:2/3).

¹H NMR (CDCl₃, 400 MHz): δ (ppm) 5.87-5.81 (m, 1H), 5.69-5.65 (m, 1H), 4.26-4.24 (m, 2H), 4.26-4.20 (m, 4H), 2.40-2.35 (m, 2H).

¹³C NMR (CDCl₃, 160 MHz): δ (ppm) 132.8, 127.5, 66.2, 61.9, 61.8, 58.7, 46.8.

¹⁹F NMR (CDC₃, C₆F₆, 376 MHz): δ (ppm) 80.9, 49.3, 44.0, 40.3, 39.8, 39.0, 35.6.

MS (ESI) m/z=535 [M+H]⁺

### Cis-1H,1H,2H,2H-perfluorodecyloxybutenyl-2,3,4,6-tetra-O-acetyl-α-D-mannopyranoside

To a solution of 2,3,4,6-tetra-O-acetyl-α/β-D-mannopyranosidetrichloroacetimidate (165.0 mg, 0.34 mmol) and cis-1H,1H,2H,2H-perfluorodecyloxybutenyl alcohol (140.0 mg, 0.25 mmol) in dichloromethane (5 mL) were added powdered 4 Å molecular sieves (10 mg) and the mixture was cooled down to 15° C. TMSOTf (40 μL, 0.17 mmol) was added and the reaction mixture was stirred at −15° C. for 30 min. The reaction was quenched with triethylamine (0.1 mL) and concentrated. The crude product was purified by solid-phase extraction using a fluorous solid-phase extraction cartridge. Non-fluorous compounds were eluted with 80% MeOH/water and the desired product was eluted by 100% MeOH. The solvent was removed under reduced pressure and 1H,1H,2H,2H-perfluorodecyloxybutenyl-2,3,4,6 tetra-O-acetyl-α-D-mannopyranoside was obtained as a colorless oil (207.1 mg, 24.0 mmol, 93%).

R_{f}: 0.26 (ethyl acetate/hexane:2/3).

¹H NMR (CDCl₃, 400 MHz): δ (ppm) 5.74-5.69 (m, 2H), 5.32 (dd, 1H, J=10.0, 3.2 Hz), 5.26 (t, 1H, J=9.6 Hz), 5.21-5.20 (m, 1H), 4.83-4.81 (m, 1H), 4.26-4.18 (m, 3H), 4.10-4.02 (m, 5H), 3.99-3.95 (m, 1H), 2.14 (s, 3H), 2.07 (s, 3H), 2.01 (s, 3H), 1.97 (s, 3H).

¹³C NMR (CDCl₃, 160 MHz): δ (ppm) 171.4, 170.9, 170.2, 170.0, 129.7, 128.9, 98.5, 71.6, 69.3, 68.5, 66.3, 66.2, 63.1, 62.4, 62.0, 21.0, 20.9(2), 20.8.

MS (ESI) m/z=887 [M+Na]⁺

1H,1H,2H,2H-perfluorodecyloxybutanyl-α-D-mannopyranoside. Cis-1H,1H,2H,2Hperfluorodecyloxybutenyl-2,3,4,6-tetra-O-acetyl-α-D-mannopyranoside (184.0 mg, 0.21 mmol) was dissolved in MeOH (5 mL) and 5% Pd/C (10 mg) was added. The reaction mixture was put under hydrogen atmosphere and stirred for 1 h. It was then filtered over Celite and ethanol was removed under reduced pressure to give the hydrogenated product in quantitative yield. To a solution of the resulting compound (184.0 mg, 21.3 mmol) in methanol (10 mL) was added K₂CO₃ (117.0 mg, 0.85 mmol) and the reaction mixture was stirred for 2 h. The mixture was then neutralized using Amberlyst-15 ion-exchange resin and filtered. The solvent was removed under reduced pressure and the desired product was obtained as a white solid (146 mg, 0.21 mmol, 100%).

R_{f}: 0.36 (methanol/ethyl acetate/hexane:2/2/3).

¹H NMR (MeOD, 400 MHz): δ (ppm) 4.71 (br, 1H), 3.80 (dd, 1H, J=12.0, 2.4 Hz), 3.75-3.72 (m, 2H), 3.70 (d, 1H, J=5.6 Hz), 3.67-3.65 (m, 1H), 3.62-3.60 (m, 2H), 3.58 3.53 (m, 2H), 3.50-3.47 (m, 2H), 3.45-3.40 (m, 1H), 2.06-2.01 (m, 2H), 1.64-1.58 (m, 4H).

¹³C NMR (MeOD, 160 MHz): δ (ppm) 100.2, 73.3, 71.3, 70.9, 70.5, 67.2, 66.9, 61.4, 61.3, 27.4 (2), 26.1(2).

MS (ESI) m/z=721 [M+Na]⁺

Cis-1H,1H,2H,2H-perfluorodecyloxybutenyl-3,4,6-tri-O-acetyl-2-deoxy-2acetylamino-β-D-glucopyranoside. To a solution of 3,4,6-tri-O-acetyl-2-deoxy-2acetylamino-α/β-D-glucopyranoside trichloroacetimidate (148.8 mg, 0.30 mmol) and cis-1H,1H,2H,2H-perfluorodecyloxybutenyl alcohol (110.0 mg, 0.25 mmol) indichloromethane (5 mL) were added powdered 4 Å molecular sieves (10 mg) and the mixture was cooled to −15° C. TMSOTf (34 μL, 0.15 mmol) was added and the reaction mixture was stirred at −15° C. for 30 min. The reaction was quenched with triethylamine (0.1 mL) and concentrated. The crude product was purified by solid-phase extraction using a fluorous solid-phase extraction cartridge. Non-fluorous compounds were eluted with 80% MeOH/water and the desired product was eluted by 100% MeOH. The solvent was removed under reduced pressure and the product was obtained as a colorless oil (150.3 mg, 0.17 mmol, 86%).

R_{f}: 0.26 (ethyl acetate/hexane:2/3).

¹H NMR (CDCl₃, 400 MHz): δ (ppm) 5.82-5.63 (m, 2H), 5.25 (dd, 1H, J=10.0, 9.6 Hz), 5.03 (t, 1H, J=10.0 Hz), 4.68 (d, 1H, J=8.4 Hz), 4.35-4.30 (m, 1H), 4.25-4.18 (m, 4H), 4.12-4.08 (m, 1H), 4.00-3.98 (m, 2H), 3.85-3.78 (m, 1H), 3.67-3.63 (m, 1H), 2.04 (s, 3H), 1.99 (s, 3H), 1.98 (s, 3H), 1.90 (s, 3H).

¹³C NMR (CDCl₃, 160 MHz): δ (ppm) 170.9, 170.7, 170.3, 169.4, 129.3, 128.9, 99.6, 72.3, 71.9, 68.6, 66.4, 64.4, 62.1, 61.9(2), 54.7, 23.3, 20.7(2), 20.6.

MS (ESI) m/z=886 [M+Na]⁺

1H,1H,2H,2H-perfluorodecyloxybutanyl-2-deoxy-2-acetylamino-β-Dglucopyranoside. Cis-1H,1H,2H,2H-perfluorodecyloxybutenyl-3,4,6-tri-O-acetyl-2deoxy-2-acetylamino-β-D-glucopyranoside (138.0 mg, 0.16 mmol) was dissolved in MeOH (5 mL) and 5% Pd/C (10 mg) was added. The reaction mixture was put under hydrogen atmosphere and stirred for 1 h. It was then filtered over Celite and ethanol was removed under reduced pressure to give the hydrogenated product in quantitative yield. To a solution of the resulting compound (138.0 mg, 0.16 mmol) in methanol (10 mL) was added K₂CO₃ (66.0 mg, 0.48 mmol) and the reaction mixture was stirred for 2 h. The mixture was then neutralized using Amberlyst-15 ion-exchange resin and filtered. The solvent was removed under reduced pressure and the product was obtained as a white solid (118.0 mg, 0.16 mmol, 100%).

R_{f}: 0.23 (methanol/ethyl acetate/hexane:2/2/3).

¹H NMR (MeOD, 400 MHz): δ (ppm) 4.41 (d, 1H, J=8.4 Hz), 3.89-3.83 (m, 2H), 3.84 (dd, 1H, J=12.4, 1.6 Hz), 3.67-3.63 (m, 2H), 3.47-3.38 (m, 5H), 3.24-3.21 (m, 2H), 2.08-2.01 (m, 2H), 1.95 (s, 3H), 1.61-1.52 (m, 4H).

¹³C NMR (MeOD, 160 MHz): δ (ppm) 172.3, 101.4, 78.2, 77.9, 77.6, 74.7, 70.7, 64.2, 61.4, 56.0, 25.9, 25.8, 21.6(2).

MS (ESI) m/z=762 [M+Na]⁺

Cis-1H,1H,2H,2H-perfluorodecyloxybutenyl-2,3,4,6-tetra-O-acetyl-β-D galactopyranoside. 2,3,4,6-Tetra-O-acetyl-α/β-D-galactopyranoside trichloroacetimidate⁸ (77.0 mg, 0.16 mmol) and cis-1H,1H,2H,2H perfluorodecyloxybutenyl alcohol (69.0 mg, 0.13 mmol) were dissolved in dichloromethane (2 mL) and the mixture was cooled to −15° C. TMSOTf (12 μL, 0.065 mmol) was added and the reaction mixture was stirred at −15° C. for 30 min. The reaction was quenched with triethylamine (0.1 mL) and concentrated. The crude product was purified by solid-phase extraction using a fluorous solid-phase extraction cartridge. Non fluorous compounds were eluted with 80% MeOH/water and the desired product was eluted by 100% MeOH. The solvent was removed under reduced pressure and the product was obtained as a colorless oil (99 mg, 0.11 mmol, 90%).

R_{f}: 0.52 (ethyl acetate/hexane:1/1)

¹H NMR (CDCl₃, 400 MHz): δ (ppm) 5.69-5.74 (m, 2H), 5.38 (d, 1H, J=3.3 Hz, H-1), 5.21 (dd, 1H, J=10.5, 2.7 Hz), 5.01 (dd, 1H, J=10.5, 3.3 Hz), 4.48 (d, 1H, J=7.8 Hz), 4.36-4.41 (m, 1H), 4.04-4.26 (m, 6H), 3.86-3.91 (m, 1H), 2.14 (s, 3H), 2.04 (s, 3H), 2.05 (s, 3H), 1.97 (s, 3H).

¹³C NMR(CDCl₃, 160 MHz): δ (ppm) 170.5, 170.4, 170.3, 169.6, 129.4, 128.9, 100.3, 71.1, 70.9, 68.9, 67.2, 66.5, 64.9, 62.1, 62.1, 61.4, 20.9, 20.8(2), 20.7.

MS (ESI) m/z=887 [M+Na]⁺

1H,1H,2H,2H-perfluorodecyloxybutanyl-β-D-galactopyranoside. Cis-1H,1H,2H,2Hperfluorodecyloxybutenyl-2,3,4,6-tetra-O-acetyl-β-D-galactopyranoside (50 mg, 0.058 mmol) was dissolved in ethanol (3 mL) and 5% Pd/C (10 mg) was added. The reaction mixture was put under hydrogen atmosphere and stirred for 1 h. It was then filtered over Celite and ethanol was removed under reduced pressure to give the hydrogenated product in quantitative yield. To a solution of the resulting compound (45 mg, 0.052 mmol) in methanol (3 mL) was added KCO₃ (4 mg, 0.03 mmol) and the reaction mixture was stirred for 2 h. The mixture was then neutralized using Amberlyst-15 ion-exchange resin and filtered. The solvent was removed under reduced pressure and the product was obtained as a white solid (40.0 mg, 0.058 mol, 100%).

R_{f}: 0.23 (methanol/ethyl acetate/hexane:2/2/3).

¹H NMR (MeOD, 400 MHz): δ (ppm) 4.17 (d, 1H, J=5.4 Hz), 3.44-3.91 (m, 12H), 2.01 (m, 2H), 1.60-1.70 (m, 4H).

¹³C NMR (MeOD, 160 MHz): δ (ppm) 103.6, 75.2, 73.7, 71.2, 69.2, 69.1, 68.9, 61.4, 61.3, 28.8 (2), 25.9(2).

MS (ESI) m/z=721 [M+H]⁺

Allyl-2-O-benzyl-3,4-di-O-carboxybenzyl-α-D-fucopyranoside. Allyl-2-O-benzyl-αD-fucopyranoside (917 mg, 3.12 mmol) was dissolved in methylene chloride (15 mL) and cooled to 0° C. TMEDA (434 mg, 3.74 mmol) and benzylchloroformate (1.20 g, 6.86 mmol) were added. The reaction mixture was stirred at 0° C. for 45 min and quenched with water (10 mL). The organic layer separated and the aqueous layer was extracted with methylene chloride (2×40 mL), washed with HCl (2N, 20 mL), brine (20 mL), and dried over MgSO₄. After removal of the solvent under reduced pressure, the crude product was purified by flash column chromatography on silica gel using 25% EtOAc/hexane as eluent. The product was obtained as a clear oil (1.7 g, 3.0 mmol, 92%).

R_{f}: 0.48 (ethyl acetate/hexane:1/1)

¹H NMR (CDCl₃, 400 MHz): δ (ppm) 7.25-7.40 (m, 15H), 5.88-5.94 (m, 1H), 5.15 5.36 (m, 8H), 4.82 (d, 1H, J=4 Hz, H-1), 4.70 (d, 1H, J=12 Hz), 4.53 (d, 1H, J=12 Hz), 4.13-4.17 (m, 2H), 3.98 (dd, 1H, J=12.8, 6.4 Hz), 3.90 (dd, 1H, J=9.6, 3.6 Hz), 1.17 (d, 3H, J=6.8 Hz).

¹³C NMR (CDCl₃, 160 MHz): δ (ppm) 155.3, 154.4, 138.2, 135.3, 135.1, 133.7, 128.6, 128.6, 128.5, 128.5, 128.4, 128.1, 127.9, 127.8, 117.9, 96.3, 75.6, 74.3, 73.7, 73.4, 69.9, 69.9, 68.6, 64.3, 15.8.

MS (ESI) m/z=585[M+Na]⁺

2-O-benzyl-3,4-di-O-carboxybenzyl-α-D-fucopyranose. Palladium chloride (685 mg, 3.87 mmol) and sodium acetate (635 mg, 7.74 mmol) were dissolved in an AcOH/water mixture (5:1, 36 mL). The mixture was heated in commercial microwave oven at 80° C. for 5 min. The reaction was then put on an 80° C. oil bath and allyl-2-O-benzyl-3,4-di-Ocarboxybenzyl-α-D-fucopyranoside (1.45 g, 2.58 mmol) in AcOH was added slowly. The reaction mixture was heated for 5 min and allowed to cool down to ambient temperature. The mixture was poured into water and extracted with ether (2×60 mL). The combined organic layer was washed with NaHCO₃ (2×40 mL), water (40 mL), and brine (20 mL) and dried over MgSO₄. The solvent was removed under reduced pressure and the crude product was purified by flash column chromatography on silica gel using 32% EtOAc/hexane as eluent. The product was obtained as a mixture of anomers (1.19 g, 2.27 mmol, 88%).

R_{f}: 0.44 (ethyl acetate/hexane:1/1)

¹H NMR (CDCl₃, 400 MHz): δ ppm) 7.15-7.36 (m, 19H), 5.12-5.22 (m, 8H), 4.81-4.90 (m, 1H), 4.50-4.74 (m, 3H), 4.31-4.49 (m, 0.5H), 3.82-4.24 (m, 3H), 1.21 (d, 0.6H, J=6.4 Hz), 1.15 (t, 3H, J=6.4 Hz).

¹³C NMR (CDCl₃, 160 MHz): δ (ppm) 137.7, 135.2, 129.2, 129.2, 128.7, 128.7, 128.7, 128.7, 128.7, 128.7, 128.6, 128.6, 128.5, 128.5, 128.5, 128.5, 128.4, 128.4, 128.3, 128.3, 128.3, 128.3, 128.2, 128.1, 128.0, 128.0, 128.0, 97.8, 91.8, 75.6, 75.5, 75.5, 75.2, 74.7, 74.5, 74.3, 74.2, 74.1, 73.9, 73.6, 73.6, 72.7, 70.2, 70.2, 70.2, 70.1, 69.1, 65.0, 64.7, 64.6, 62.6, 43.4, 26.8, 21.9, 16.1, 16.0.

MS (ESI) m/z=523 [M+H]⁺

2-O-benzyl-3,4-di-O-carboxybenzyl-α/β-D-fucopyranoside trichloroacetimidate. To a solution of 2-O-benzyl-3,4-di-O-carboxybenzyl-α/β-D-fucopyranose (1.17 g, 2.24 mmol) in dichloromethane (10 mL) were added powdered 4 Å molecular sieves (100 mg) and trichloroacetonitrile (2.2 mL, 22.4 mmol). The reaction was stirred for 30 min and Cs₂CO₃ (802 mg, 2.46 mmol) was added. The reaction mixture was further stirred for 45 min and filtered over Celite. The eluent was concentrated and the crude product was purified by flash column chromatography on silica gel using 28% EtOAc/hexane as eluent to provide the product as a white solid (1.43 g, 2.1 mmol, 92%).

R_{f}: 0.46 (ethyl acetate/hexane:1/1)

¹H NMR (CDCl₃, 400 MHz): δ (ppm) 8.68 (s, 1H), 7.19-7.39 (m, 15H), 5.78 (d, 1H, J=8.1 Hz), 5.15-5.29 (m, 6H), 4.96 (dd, 1H, J=9.9, 3.3 Hz), 4.83 (d, 1H, J=11.0 Hz), 4.67 (d, 1H, J=11.0 Hz), 3.94-4.00 (m, 1H), 1.27 (d, 3H, J=6.3 Hz).

¹³C NMR (CDCl₃, 160 MHz): δ(ppm) 161.3, 155.4, 154.5, 137.9, 135.2, 135.1, 128.8, 128.8, 128.8, 128.81, 128.8, 128.5, 128.5, 128.4, 127.9, 127.9, 98.1, 77.1, 75.6, 75.4, 74.5, 70.4, 70.3. 69.9, 16.2.

MS (ESI) m/z=667 [M+H]⁺

Cis-1H,1H,2H,2H-perfluorodecyloxybutenyl-2-O-benzyl-3,4-di-O-carboxybenzyl-αD-fucopyranoside. To a solution of 2-O-benzyl-3,4-di-O-carboxybenzyl-α/β-Dfucopyranoside trichloroacetimidate (185.0 mg, 0.28 mmol) and cis-1H,1H,2H,2Hperfluorodecyloxybutenyl alcohol (123.0 mg, 0.23 mmol) in dichloromethane (5 mL) were added powdered 4 Å molecular sieves (10 mg) and the mixture was cooled down to 15° C. TMSOTf (42 μL, 0.23 mmol) was added and the reaction mixture was stirred at −15° C. for 30 min. The reaction was quenched with triethylamine (0.1 mL) and concentrated. The crude product was purified by solid-phase extraction using a fluorous solid-phase extraction cartridge. Non-fluorous compounds were eluted with 80% MeOH/water and the desired product was eluted by 100% MeOH. The solvent was removed under reduced pressure and the product was obtained as a colorless oil (215 mg, 20.7 mmol, 90%).

R_{f}: 0.26 (ethyl acetate/hexane:2/3).

¹H NMR (CDCl₃, 400 MHz): δ (ppm) 7.26-7.36 (m, 15H), 5.72-5.84 (m, 2H), 5.15-5.23 (m, 6H), 4.44-4.81 (m, 4H), 4.05-4.23 (m, 6H), 3.66-3.78 (m, 1H), 1.15-1.27 (dd, 3H, J=6.4 Hz).

¹³C NMR (CDCl₃, 160 MHz): δ (ppm) 155.3, 154.4, 138.1, 135.2, 135.1, 128.9, 128.7, 128.6, 128.6, 128.6, 128.4, 128.3, 128.3, 128.2, 128.0, 127.8, 127.7 (2), 96.5, 73.3, 75.5, 74.2, 73.6, 73.5, 70.0, 69.9, 66.4, 64.4, 63.2, 61.9 (2), 26.12), 15.7.

MS (ESI) 171/z=1061 [M+Na]⁺

1H,1H,2H,2H-perfluorodecyloxybutanyl-α-D-fucopyranoside. Cis-1H,1H,2H,2Hperfluorodecyloxybutenyl-2-O-benzyl-3,4-di-O-carboxybenzyl-α-D-fucopyranoside (81 mg, 0.078 mmol) was dissolved in ethanol (3 mL) and 5% Pd/C (20 mg) was added. The reaction mixture was put under hydrogen atmosphere and stirred overnight. The mixture was then filtered over Celite and ethanol was removed under reduced pressure to give the hydrogenated product in quantitative yield (81 mg, 0.078 mmol, 100%).

R_{f}: 0.28 (methanol/ethyl acetate/hexane:2/2/3).

¹H NMR (MeOD, 400 MHz): δ (ppm) 4.15-4.31 (m, 1H), 3.45-3.98 (m, 1H), 1.62-1.68 (Sn, 4H), 1.23 (dd, 3H, J=16.8, 6.4 Hz).

¹³C NMR (MeOD, 160 MHz): δ (ppm) 99.1, 72.3, 70.6, 70.3, 68.6, 67.4, 66.1, 64.3 (2), 26.1(2), 15.2.

MS (ESI) m/z=705 [M]+Na]⁺

### EXAMPLE 4

### Synthesis of Carbohydrate Microarrays

This study describes the tagging of rabinose, rhamnose, lactose, maltose, and glucosamine with a single C₈F₁₇-tail, incorporation of these tags into expanded carbohydrate microarrays, and screening of this microarray against two lectins to test the scope of a fluorous-based microarray method.

To obtain a flexible glucosamine building block, the N-p-nitrobenzyloxycarbonyl-protected glucosamine donor 12 was selected. This donor can be readily obtained, easily deprotected by hydrogenolysis to provide a free amine, and also transformed directly to the desired N-acetamido modified analog in one pot by addition of acetic anhydride during the hydrogenolysis reaction. Thus, the glycosylation reaction of 3,4,6-tri-O-acetyl-2-deoxy-2(p-nitronitrobenzyloxycarbonylamino)-α/β-D-glucopyranosyl trichloroacetimidate (12) with fluorous alcohol with trimethylsilyl triflate as an activator gave the desired fluorous-tagged compound (13) in 84% yield. Compound 13 was deacetylated under basic conditions to give compound 14, which upon hydrogenolysis in methanol provided the 3-(perfluorooctyl)propanyloxybutanyl-2-deoxy-2-amino-β-D-glucopyranoside (4) in 94% yield (Scheme 2). Alternately, the hydrogenolysis of 14 when performed using acetic anhydride in methanol resulted in the formation of 3-(perfluorooctyl)propanyloxybutanyl-2-deoxy-2-acylamino-β-D-glucopyranoside (5) in 82% (Scheme 2).

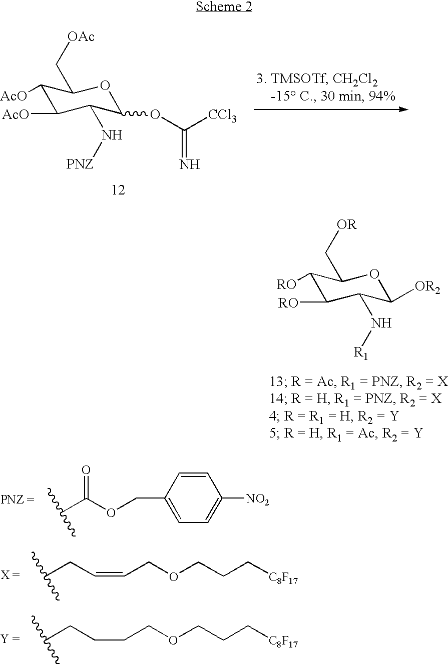

Similarly, the glycosylation reactions of fluorous alcohol 3 were performed with the known tricholoroacetimidate donors of D-mannose 15 [19], D-galactose 16 [20], L-arabinose 17 [21], L-rhamnose 18 [22], D-lactose 19 [23], and D-maltose 20 [24] to provide fluorous-tagged glycosides 21-26. Alkene hydrogenation of all the intermediates followed by global deacylation resulted in the formation of the desired compounds 6-11 (Scheme 3) for formation of microarrays.

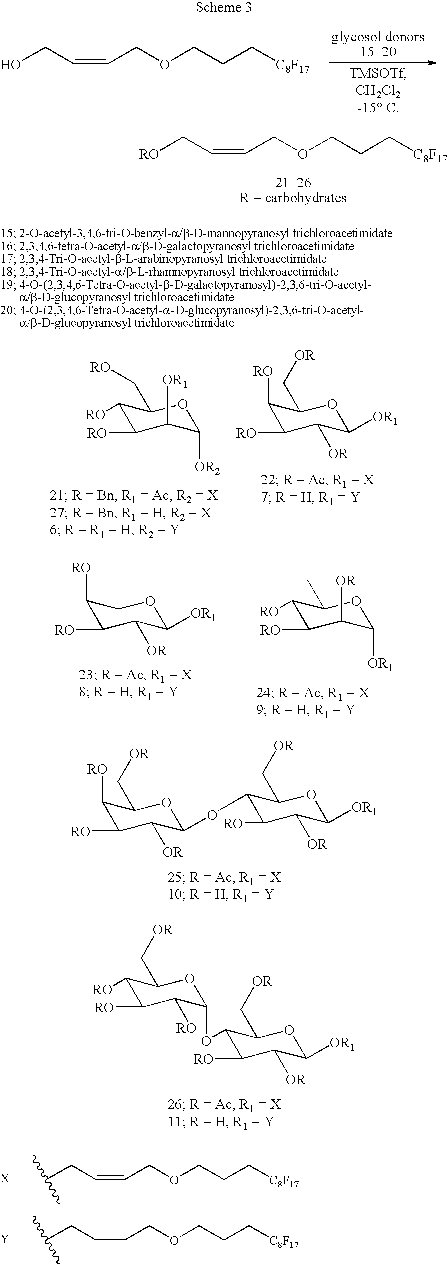

With the fluorous-tagged carbohydrates in hand, the ability of the single C₈F₁₇ tail to anchor these mono- and disaccharides to a glass slide surface even after repeated washes was examined. The fluorous-tagged carbohydrates 4-11 were dissolved in 80% methanol/water and were spotted onto fluoroalkylsilane-derivatized glass slides employing a standard robot used for DNA arraying. The slides then were incubated for 30 minutes with the commercially available FITC (fluorescein isothiocyanate)-labeled lectins from Triticum vulgaris (wheat germ, FITC-WGA) in phosphate buffered saline (PBS buffer) (0.25 M) and Arachis hypogaea (peanut, FITC-PNA) in PBS buffer (0.5 M) and rinsed repeatedly with the assay buffer followed by distilled water. After drying, the slide was scanned with a standard fluorescent slide scanner at 488 nm to reveal the carbohydrate-binding specificities of these two lectins. As expected the wheat germ lectin WGA bound only to the fluorous-tagged N-acetylglucosamine (5). In contrast, the peanut lectin PNA bound to both galactose (7) and lactose (10) in the array experiment. This lectin is known to bind to galactose both alone and in the context of the lactose disaccharide. This experiment clearly shows that the disaccharide is retained on the fluorous-derivatized glass slides under the conditions required for screening of proteins for their carbohydrate-binding specificities.

In conclusion, the new method described herein for the facile fabrication of carbohydrate microarrays by using the noncovalent immobilization of carbohydrates on fluorous derivatized glass slides clearly can be extended beyond monosaccharides. Screening of these carbohydrate microarrays against two lectins demonstrates that the noncovalent fluorous-fluorous interaction is sufficient to retain not only mono- but also disaccharides under biological assay conditions. As the fluorous tag also facilitates purification during synthesis, this microarray approach should be particularly valuable for screening of synthetic carbohydrate libraries.

Experimental

General Experimental Procedures.

Dichloromethane was distilled from calcium hydride before use. Amberlyst 15 ion-exchange resin was washed repeatedly with methanol before use. All other commercial reagents and solvents were used as received without further purification.

The reactions were monitored and the R_{f} values determined using analytical thin layer chromatography (tic) with 0.25 mm EM. Science silica gel plates (60F-254). The developed tlc plates were visualized by immersion in p-anisaldehyde solution followed by heating on a hot plate. Flash chromatography was performed with Selecto Scientific silica gel, 32-63 μm particle size. Fluorous phase chromatography was performed using fluorous solid-phase extraction cartridges containing silica gel bonded with perfluorooctylethylsilyl chains (Fluorous Technologies, Inc.; Pittsburgh, Pa.). All other fluorous reagents were also obtained from Fluorous Technologies, Inc. All moisture-sensitive reactions were performed in flame- or oven-dried glassware under a nitrogen atmosphere. Bath temperatures were used to record the reaction temperature. All reactions were stirred magnetically at ambient temperature unless otherwise indicated.

¹H NMR, and ¹³C NMR spectra were obtained with a Bruker DRX400 at 400 MHz, 100 MHz, and 162 MHz respectively. ¹H NMR spectra were reported in parts per million (δ) relative to CDCl₃ (7.27 ppm) and CD₃OD (4.80) as an internal reference. ¹³C NMR spectra were reported in parts per million (δ) relative to CDCl₃ (77.23 ppm) or CD₃OD (49.15 ppm).

Microarray Preparation and Screening.

Fluorous-tagged carbohydrate compounds were dissolved in 80% methanol/water (2 mM) and spotted on clean fluorinated glass slides using a robotic spotter (Cartesian PixSys 5500 Arrayer, Cartesian Technologies, Inc., Irvine, Calif.) at 30% humidity. The glass slide was dried in a humidifying chamber at 30% humidity for 2 h.

FITC-labeled PNA or WGA (EY Laboratories, San Mateo, Calif.) in PBS buffer (pH=7.3-7.4, 2.35 mM CaCl₂, 0.85 nM MgCl₂, 114 mM NaCl, 4 mM KCl, 1 mM Na₂HPO₄, 15 mM NaHCO₃, 11.1 mM glucose; 0.5 M for PNA and 0.25 M for WGA assay) were used for the detection of protein-carbohydrate interactions. The arrays were incubated with the protein solution (0.1 mL) by using a PC500 CoverWell incubation chamber (Grace Biolabs, Bend, Oreg.) and gently shaken every 5 min for 30 min. The slides were then washed three times with the incubation buffer followed by three washes with distilled water. The slides were subsequently dried for 30 min in a dark humidity chamber. The glass slides were scanned using a General Scanning ScanArray 5000 set at 488 nm.

### Synthesis of 3-(perfluorooctyl)propanyl methyl sulfonate (2)

To a solution of 3-(perfluorooctyl)propanol 1 (2.0 g, 4.2 mmol) in dichloromethane (20 mL) was added triethylamine (1.20 mL, 8.36 mmol) and the mixture was cooled to 0° C. Mesyl chloride (0.64 mL, 8.4 mmol) was added dropwise over 5 min and the reaction was allowed to warm to ambient temperature over 2 h. The reaction was diluted with dichloromethane (100 mL), washed with water (40 mL), HCl (2N, 40 mL), and brine (30 mL), and dried over MgSO₄. The solvent was removed under reduced pressure to obtain 2 (2.32 g, 100%) as a solid.

¹H NMR (300 MHz, CDCl₃): δ 2.05-2.13 (m, 2H), 2.20-2.36 (m, 2H), 3.04 (s, 3H), 4.31 (t, J=6 Hz, 2H)

### Synthesis of 3-(perfluorooctyl)propanyloxybutenylalcohol (3)

To a solution of cis-1,4-butenediol (0.48 g, 5.4 mmol), 3-(perfluorooctyl)propanyl methyl sulfonate (2.3 g, 4.16 mmol), and tetrabutylammonium bromide (0.27 g, 0.83 mmol) in DMF (20 mL was added powdered KOH (0.47 g, 8.32 mmol). The reaction mixture was heated at 70° C. for 1 h and then poured into water (20 mL). The aqueous layer was extracted with ethyl acetate (2×60 mL), washed with water (30 mL) and brine (30 mL), and dried over MgSO₄. The solvent was removed under reduced pressure. The crude product was purified by flash column chromatography on silica gel using 27% EtOAc/hexane as eluent to obtain 3 (1.57 g, 70%) as a yellow oil.

¹H NMR (300 MHz, CDCl₃): δ 1.78-1.88 (m, 2H), 2.04-2.19 (m, 2H), 2.99 (s, 1H), 3.45 (t, J=6.0 Hz, 2H), 4.0 (d, J=6.3 Hz, 214), 4.13 (d, J=6.3 Hz, 2H), 5.57-5.65 (m, 1H), 5.70-5.78 (m, 1H). ¹³C NMR (75, MHz, CDCl₃): δ 20.8, 28.0, 58.4, 66.4, 68.9, 127.9, 132.3.

### Synthesis of 3-(perfluorooctyl)propanyloxybutenyl-3,4,6-tri-O-acetyl-2-deoxy-2(p-nitrobenzyloxycarbonylamino)-β-D-glucopyranoside (13)

To a solution of 3,4,6-tri-O-acetyl-2-deoxy-2(p-nitronitrobenzyloxycarbonylamino)-α/β-D-glucopyranosyl trichloroacetimidate (12) (1.68 g, 2.7 mmol) and 3-(perfluorooctyl) propanyloxybutenyl alcohol (3) (1.0 g, 1.8 mmol) in dichloromethane (20 mL) was added TMSOTf (0.16 mL, 0.9 mmol) at −15° C. The reaction mixture was stirred at −15° C. for 30 min. The reaction was quenched with triethylamine (0.2 mL) and concentrated. The crude product was purified by solid-phase extraction using a fluorous solid-phase extraction cartridge. Nonfluorous compounds were eluted with 80% MeOH/water and the desired product was eluted by 100% MeOH. The solvent was removed under reduced pressure to obtain 13 (1.53 g, 84%) as a solid.

¹H NMR (300 MHz, CDCl₃): δ 1.50-2.40 (m, 4H), 1.98, 2.02, 2.08 (3s, 9H), 3.50 (t, J=6.0 Hz, 2H), 4.00 (d, 1H, J=5.0 Hz, 2H), 4.04-4.40 (m, 4H), 4.66 (s, 1H), 4.94 (s, 1H), 5.06 (t, J=9.4 Hz, 1H), 5.14-5.30 (m, 3H), 5.50-5.70 (m, 2H), 7.48 (d, J=8.3 Hz, 2H), 8.20 (d, J=8.8 Hz, 2H). ¹³C NMR (75 MHz, CDCl₃): δ 20.6, 20.7, 20.8, 20.9, 28.0, 56.3, 62.3, 65.4, 66.5, 68.9, 69.0, 71.9, 72.3, 99.7, 123.8, 128.0, 130.5, 144.1, 147.7, 155.6, 169.6, 170.8. MS (ESI) m/z 1037 (M+Na)⁺.

### Synthesis of 3-(perfluorooctyl)propanyloxybutenyl-2-deoxy-2(p-nitrobenzyloxycarbonylamino)-β-D-glucopyranoside (14)

To a solution of 3-(perfluorooctyl)propanyloxybutenyl-3,4,6-tri-O-acetyl-2-deoxy-2(p-nitrobenzyloxycarbonylamino)-β-D-glucopyranoside (13) (1.4 g, 1.4 mmol) in methanol (15 mL) was added NaOMe (70 mg); the reaction mixture was stirred at ambient temperature for 3 h. The reaction was neutralized with Amberlyst-15 ion-exchange resin and filtered. The solvent was removed under reduced pressure to obtain 14 (1.2 g, 98%) as a solid.

¹H NMR (300 MHz, CD₃OD): δ 1.70-1.90 (m, 2H), 2.10-2.40 (m, 2H), 3.10-3.50 (m, 7H), 3.54-3.66 (m, 1H), 3.90 (d, J=10.4 Hz, 1H), 4.06 (d, J=5.0 Hz, 1H), 4.20-4.50 (m, 3H), 5.10-5.40 (m, 2H) 5.50-5.80 (m, 2H), 7.60 (d, J=8.2 Hz, 2H), 8.20 (d, J=8.5 Hz, 2H). ¹³C NMR (75 MHz, CDCl₃): δ 21.0, 28.0, 58.1, 61.9, 64.7, 65.1, 66.7, 68.8, 71.3, 75.1, 77.2, 101.0, 123.6, 128.1, 128.6, 130.0, 145.4, 147.9, 157.7, 169.5. MS (ESI) m/z 911 (M+Na)⁺.

### 4.7 Synthesis of 3-(perfluorooctyl)propanyloxybutanyl-2-deoxy-2-amino-β-D-glucopyranoside (4)

To a solution of 3-(perfluorooctyl)propanyloxybutenyl-2-deoxy-2(p-nitrobenzyloxycarbonylamino)-β-D-glucopyranoside (14) (60 mg, 0.07 mmol) in methanol (2 mL) was added 5% Pd/C (7 mg). The reaction mixture was stirred at ambient temperature under hydrogen atmosphere for 2 h. The reaction was then filtered over Celite and the solvent was removed under reduced pressure to obtain 4 (45 mg, 94%) as a solid.

¹H NMR (300 MHz, CD₃OD): δ 1.60-1.90 (m, 6H), 2.10-2.40 (m, 2H), 2.50-2.80 (m, 2H), 3.30-3.70 (m, 12H), 3.86 (d, J=11.4 Hz, 1H), 3.92-4.08 (m, 1H), 4.34 (d, J=8.2 Hz, 1H). ¹³C NMR (100 MHz, CD₃OD): δ 20.7, 26.2, 26.3, 27.7, 57.2, 61.5, 68.8, 69.3, 70.5, 70.6, 76.5, 76.9, 103.5. MS (ESI) m/z 734 (M+Na)⁺.

### 4.8 Synthesis of 3-(perfluorooctyl)propanyloxybutanyl-2-deoxy-2-acylamino-β-D-glucopyranoside (5)

To a solution of 3-(perfluorooctyl)propanyloxybutenyl-2-deoxy-2(p-nitrobenzyloxycarbonylamino)-β-D-glucopyranoside (14) (40 mg, 0.04 mmol) in methanol (0.5 mL) and acetic anhydride (1.0 mL) was added 5% Pd/C (4 mg). The reaction mixture was stirred at ambient temperature under hydrogen atmosphere for 13 h. The reaction mixture was then filtered over Celite and the solvent was removed under reduced pressure to obtain 5 (28 mg, 82%) as a solid.

¹H NMR (300 MHz, CD₃OD): δ 1.40-1.74 (m, 4H), 1.80-1.90 (m, 2H), 1.98 (s, 3H), 2.10-2.40 (m, 4H), 3.20-3.80 (m, 14H), 3.80-4.10 (m, 2H), 4.38 (d, J=8.4 Hz, 1H). ¹³C NMR (100 MHz, CD₃OD): δ 20.7, 21.8, 26.0, 26.1, 26.2, 27.7, 29.2, 56.2, 61.6, 61.7, 68.8, 68.9, 69.1, 70.5, 70.6, 70.9, 74.9, 76.8, 101.5, 172.8.

### 4.9 Synthesis of 3-(perfluorooctyl)propanyloxybutenyl-2-O-acetyl-3,4,6-O-benzyl-α-D-mannopyranoside (21)

To a solution of 2-O-acetyl-3,4,6-O-benzyl-α/β-D-mannopyranosyl trichloroacetimidate (15) (0.37 g, 0.58 mmol) and 3-(perfluorooctyl)propanyloxybutenyl alcohol (3)(0.21 g, 0.38 mmol) in dichloromethane (4 mL) was added TMSOTf (14 μL, 0.077 mmol) at 5° C. The reaction mixture was stirred at 5° C. for 30 min. The reaction was quenched with triethylamine (0.05 mL) and concentrated. The crude product was purified by solid-phase extraction using a fluorous solid-phase extraction cartridge. Nonfluorous compounds were eluted with 80% MeOH/water and the desired product was eluted by 100% MeOH. The solvent was removed under reduced pressure to obtain 21 (0.36 mg, 92%) as a viscous yellow oil.

¹H NMR (300 MHz, CDCl₃): δ 1.80-1.88 (m, 2H), 2.11-2.27 (m, 2H) (s, 3H), 3.43 (t, J=6.0 Hz, 2H), 3.75-3.92 (m, 4H), 4.01-4.04 (m, 3H), 4.07-4.15 (m, 1H), 4.19-4.26 (m, 1H), 4.45-4.58 (m, 31-1), 4.70 (dd, J=9.9 Hz, 12.0 Hz, 2H), 4.88 (dd, J=6.6 Hz, 8.4 Hz, 2H), 5.35-5.41 (m, 1H), 5.64-5.77 (m, 2H), 7.15-7.36 (m, 15H). ¹³C NMR (75 MHz, CDCl₃): δ 20.1, 61.7, 65.4, 67.6, 67.7, 67.9, 70.5, 70.7, 72.4, 73.3, 74.2, 77.2, 95.8, 126.6, 126.7, 126.8, 126.8, 126.9, 126.9, 127.0, 127.3, 127.4, 129.3, 136.9, 137.1, 137.2, 169.4.

### 4.10 Synthesis of 3-(perfluorooctyl)propanyloxybutenyl-3,4,6-O-benzyl-α-D-mannopyranoside (27)

To a solution of 3-(perfluorooctyl)propanyloxybutenyl-2-O-acetyl-3,4,6-O-benzyl-α-D-mannopyranoside (21) (0.36 g, 0.352 mmol) in methanol (4 mL) was added NaOMe (40 mg) and the reaction mixture was stirred at ambient temperature for 1 h. The reaction was neutralized with Amberlyst-15 ion-exchange resin and filtered. The solvent was removed under reduced pressure to obtain 27 (0.35 g, 100%) as a yellow oil.

¹H NMR (400 MHz, CDCl₃): δ 1.78-1.87 (m, 2H), 2.08-2.23 (m, 2H), 2.53 (s, 1H), 3.40 (t, J=6.0 Hz, 2H), 3.68-3.92 (m, 5H), 4.0-4.12 (m, 4H), 4.18-4.24 (m, 1H), 4.50 (d, J=10.8 Hz, 1H), 4.53-4.65 (m, 2H), 4.68 (d, J=2.4 Hz, 2H), 4.82 (d, J=10.8 Hz, 1H), 4.92 (d, J=1.6 Hz, 1H), 5.60-5.75 (m, 2H), 7.15-7.34 (m, 15H). ¹³C NMR (100 MHz, CDCl₃): δ 20.8, 28.0, 62.5, 66.4, 68.3, 68.7, 69.0, 71.2, 72.0, 73.5, 74.3, 75.2, 80.2, 98.3, 127.6, 127.7, 127.8, 128.0, 128.1, 128.3, 128.4, 128.6, 130.2, 137.9, 138.2.

### 4.11 Synthesis of 3-(perfluorooctyl)propanyloxybutanyl-α-D-mannopyranoside (6)

To a solution of 3-(perfluorooctyl)propanyloxybutenyl-3,4,6-O-benzyl-α-D-mannopyranoside (27) (60 mg, 0.061 mmol) in methanol (3 mL) was added 10% Pd/C (20 mg). The reaction mixture was stirred at ambient temperature under hydrogen atmosphere for 12 h. The reaction mixture was then filtered over Celite and the solvent was removed under reduced pressure to obtain 6 (44 mg, 100%) as a solid.

¹H NMR (400 MHz, CD₃OD): δ 1.60-1.69 (m, 4H), 1.80-1.89 (m, 2H), 2.18-2.33 (m, 2H), 3.40-3.88 (m, 12H), 4.74 (m, 1H). ¹³C NMR (100 MHz, CD₃OD) δ 20.8, 26.2, 26.4, 27.6, 61.6, 67.1, 67.4, 68.8, 70.5, 71.1, 71.4, 73.5, 101.2.

### 4.12 Synthesis of 3-(perfluorooctyl)propanyloxybutenyl-2,3,4,6-tetra-O-acetyl-β-D-galactopyranoside (22)

To a solution of 2,3,4,6-tetra-O-acetyl-α/β-D-galactopyranosyl trichloroacetimidate (16) (100 mg, 0.20 mmol) and 3-(perfluorooctyl)propanyloxybutenyl alcohol (3) (75 mg, 0.14 mmol) in dichloromethane (3 mL) was added TMSOTf (5 μL, 0.027 mmol) at −15° C. The reaction mixture was stirred at −15° C. for 30 min. The reaction was quenched with triethylamine (0.05 mL) and concentrated. The crude product was purified by solid-phase extraction using a fluorous solid-phase extraction cartridge. Nonfluorous compounds were eluted with 80% MeOH/water and the desired product was eluted by 100% MeOH. The solvent was removed under reduced pressure to obtain 22 (97 mg, 80%) as a syrup.

¹H NMR (400 MHz, CDCl₃,): δ 1.82-1.89 (m, 2H), 1.95, 2.02, 2.03 (3s, 9H), 2.08-2.24 (m, 2H) (s, 3H), 3.46 (t, J=6.0 Hz, 2H), 3.86 (t, J=5.2 Hz, 1H), 4.01 (t, J=5.2 Hz, 1H), 4.10-4.23 (m, 4H), 4.34 (dd, J=5.6, 10.0 Hz, 1H), 4.75 (d, J=8 Hz, 1H), 4.99 (dd, J=3.6, 10.4 Hz, 1H), 5.19 (dd, J=8.0, 10.4 Hz, 1H), 5.36 (d, J=3.6 Hz, 1H), 5.60-5.76 (m, 2H). ¹³C NMR (100 MHz, CDCl₃): δ 20.5, 20.6, 20.7, 20.8, 20.9, 61.2, 64.7, 66.5, 67.0, 68.7, 68.8, 70.7, 70.9, 76.7, 100.1, 127.8, 130.2, 169.4, 170.1, 170.2, 170.3.

### 4.13 Synthesis of 3-perfluorooctyl)propanyloxybutanyl-β-D-galactopyranoside (7)

To a solution of 3-(perfluorooctyl)propanyloxybutenyl-2,3,4,6-tetra-O-acetyl-β-D-galactopyranoside (22) (97 mg, 0.110 mmol) in methanol (3 mL) was added 5% Pd/C (25 mg). The reaction mixture was stirred at ambient temperature under hydrogen atmosphere for 2 h. The reaction mixture was then filtered over Celite and the solvent was removed under reduced pressure. The product was used directly in the next step. To a solution of 3-(perfluorooctyl)propanyloxybutanyl-2,3,4,6-tetra-O-acetyl-β-D-galactopyranoside (97 mg, 0.110 mmol) in methanol (3 mL) was added NaOMe (15 mg) and the reaction mixture was stirred at ambient temperature for 2 h. The reaction was neutralized with Amberlyst-15 ion-exchange resin and filtered. The solvent was removed under reduced pressure to obtain 7 (75 mg, 98%) as a solid.

¹H NMR (300 MHz, CD₃OD): δ 1.62-1.71 (m, 4H), 1.79-1.87 (m, 2H), 2.11-2.36 (m, 2H), 3.22-3.34 (m, 1H), 3.41-3.63 (m, 6H), 3.70-3.74 (m, 3H), 3.81-3.99 (m, 2H), 4.20 (d, J=6.9 Hz, 1H). ¹³C NMR (75 MHz, CD₃OD): δ 26.1, 26.2, 26.3, 53.3, 61.9, 69.2, 69.3, 69.4, 71.1, 72.6, 74.1, 76.2, 114.4.

### 4.14 Synthesis of 3-(perfluorooctyl)propanyloxybutenyl-2,3,4-tri-O-acetyl-α-L-arabinopyranoside (23)

To a solution of 2,3,4-tri-O-acetyl-α/β-L-arabinosyl tricholoroacetimidate (17) (0.27 g, 0.64 mmol) and 3-(perfluorooctyl)propanyloxybutenyl alcohol (3)(0.15 g, 0.28 mmol) in dry dichloromethane (5 mL) was added TMSOTf (0.025 mL, 0.14 mmol) at ambient temperature. The reaction mixture was stirred at ambient temperature for 15 min. The reaction was quenched with triethylamine (0.5 mL) and concentrated. The crude product was purified by solid phase extraction using a fluorous solid-phase extraction cartridge. Nonfluorous compounds were eluted with 50% MeOH/water and the desired product was eluted by 100% MeOH. The solvent was removed under reduced pressure to obtain 23 (0.2 g, 89%) as a solid.

¹H NMR (300 MHz, CDCl₃): δ 1.80-1.95 (m, 2H), 2.0-2.3 (m, 2H), 2.00, 2.02, 2.11 (3s, 9H), 3.46 (t, J=6.0 Hz, 2H), 3.61 (d, J=1.9 Hz, 1H), 3.97-4.03 (m, 3H), 4.20 (dd, J=6.3, 12.6 Hz 1H), 4.36 (dd, J=5.7, 12.6 Hz, 1H), 4.41 (d, J=6.9 Hz, 1H), 5.02 (dd, J=3.6, 9.3 Hz, 1H), 5.13 (dd, J=4.2, 6.6 Hz, 1H), 5.23 (m, 1H), 5.60-5.75 (m, 2H). ¹³C NMR (75 MHz, CDCl₃): δ 20.9, 21.0, 21.1, 19.9, 61.6, 64.7, 66.7, 67.8, 69.0, 69.3, 70.3, 91, 92, 100.0, 128.2, 130.1, 169.7, 170.4, 170.6.

### 4.15 Synthesis of 3-(perfluorooctyl)propanyloxybutanyl-α-L-arabinopyranoside (8)

To a solution of 3-(perfluorooctyl)propanyloxybutenyl-2,3,4-tri-O-acetyl-α-L-arabinopyranoside (23) (0.1 g, 0.12 mmol) in methanol (10 mL) was added 5% Pd/C (0.25 g). The reaction mixture was stirred at ambient temperature under hydrogen atmosphere for 2 h. The reaction mixture was then filtered over Celite and partially concentrated under reduced pressure. To this solution, K₂CO₃ (67 mg) was added and the mixture was stirred at ambient temperature for 3 h. The reaction mixture was neutralized with Amberlyst-15 ion-exchange resin and filtered over Celite. The solvent was removed under reduced pressure to obtain 8 (68 mg, 80%) as a solid.

¹H NMR (300 MHz, CD₃OD): δ 1.80-1.95 (m, 2H), 2.1-2.3 (m, 2H), 3.4-3.9 (m, 14H), 4.17(d, J=4.8 Hz, 1H). ¹³C NMR (75 MHz, CD₃OD): δ 13.2, 19.6, 20.6, 21.5, 48.8, 56.7, 60.3, 66.7, 67.8, 69.0, 69.3, 102.8. MS (ESI) m/z 682, M+H)⁺.

### 4.16 Synthesis of 3-(perfluorooctyl)propanyloxybutenyl-2,3,4-tri-O-acetyl-α-L-rhamnopyranoside (24)

To a solution of 2,3,4-tri-O-acetyl-α/β-L-rhamnopyranosyl tricholoroacetimidate (18) (0.2 g, 0.46 mmol) and 3-(perfluorooctyl)propanyloxybutenyl alcohol (3) (0.13 g, 0.23 mmol) in dry dichloromethane (5 mL) was added TMSOTf (0.02 mL, 0.11 mmol) at ambient temperature. The reaction mixture was stirred for 15 min. The reaction was quenched with triethylamine (0.5 mL) and concentrated under reduced pressure. The crude product was purified by solid phase extraction using a fluorous solid-phase extraction cartridge. Nonfluorous compounds were eluted with 50% MeOH/water and the desired product was eluted by 100% MeOH. The solvent was removed under reduced pressure to obtain 24 (0.15 g, 76%) as a solid.

¹H NMR (300 MHz, CDCl₃): δ 1.24 (d, J=6.3, 3H), 1.80-1.95 (m, 2H), 2.0-2.3 (m, 2H), 1.98, 2.04, 2.14 (3s, 9H), 3.46 (t, J=6.0 Hz, 2H), 3.9 (m, 1H), 4.05 (d, J=5.1 Hz, 2H), 4.06 (d, J=6.9 Hz, 1H), 4.15 (dd, J=4.9, 11.7 Hz, 1H), 4.74 (d, J=3.0 Hz, 1H), 5.06 (t, J=9.9 Hz, 1H), 5.21 (m, 1H), 5.28 (dd, J=0.2, 3.6 Hz, 0.2 1H), 5.65-5.8 (m, 2H). ¹³C NMR (75 MHz, CDCl₃): δ 17.6, 20.9, 21, 21.01, 21.1, 63.3, 66.6, 66.7, 69.3, 69.5, 70.5, 71.2, 97, 128, 130.5, 170.2, 170.4.

### 4.17 Synthesis of 3-(Perfluorooctyl)propanyloxybutanyl-α-L-rhamnopyranoside (9)

To a solution of 3-(perfluorooctyl)propanyloxybutenyl-2,3,4-tri-O-acetyl-α-L-rhamnopyranoside (24) (0.1 g, 0.12 mmol) in methanol (5 mL) was added 5% Pd/C (250 mg). The reaction mixture was stirred at ambient temperature under hydrogen atmosphere for 2 h. It was then filtered over Celite and partially concentrated. To the solution, K₂CO₃ (66 mg) was added and the mixture was stirred at ambient temperature for 3 h. The reaction mixture was neutralized with Amberlyst-15-ion-exchange resin and filtered over Celite. The solvent was removed under reduced pressure to obtain 9 (70 mg, 82%) as a solid.

¹H NMR (300 MHz, CD₃OD): δ 1.20 (d, J=6.3 Hz, 3H), 1.80-1.95 (m, 2H), 2.1-2.3 (m, 2H), 2.9 (d, J=4.8 Hz, 1H), 3.0 (d, J=7.2, 1H), 3.30-3.70 (m, 11H), 3.75 (m, 1H), 4.65 (m, 1H). ¹³C NMR (75 MHz, CD₃OD): δ 16.7, 26.2, 26.4, 67.2, 68.6, 68.9, 70.5, 71.1, 71.2, 72.7, 100.4; MS (ESI) m/z 696 (M+H)⁺.

### 4.18 Synthesis of 3-(perfluorooctyl)propanyloxybutenyl-4-O-(2,3,4,6-tetra-O-acetyl-β-D-galactopyranosyl)-2,3,6-tri-O-acetyl-β-D-glucopyranoside (25)

To a solution of 4-O-(2,3,4,6-tetra-O-acetyl-β-D-galactopyranosyl)-2,3,6-tri-O-acetyl-β-D-glucopyranosyl trichloroacetimidate (19) (0.17 g, 0.22 mmol) and 3-(perfluorooctyl)propanyloxybutenyl alcohol (3) (0.1 g, 0.17 mmol) in dichloromethane (5 mL) was added TMSOTf (0.16 mL, 0.9 mmol) at −15° C. The reaction mixture was stirred at −15° C. for 30 min. The reaction was quenched with triethylamine (0.2 mL) and concentrated. The crude product was purified by solid-phase extraction using a fluorous solid-phase extraction cartridge. Nonfluorous compounds were eluted with 80% MeOH/water and the desired product was eluted by 100% MeOH. The solvent was removed under reduced pressure to obtain 25 (0.12 g, 52%) as a solid.

¹H NMR (400 MHz, CDCl₃): δ 1.80-1.90 (m, 2H), 1.93, 1.98, 2.00, 2.02, 2.08, 2.11 (7s, 21H), 1.90-2.30 (m, 2H), 3.44 (t, J=6.0 Hz, 2H), 3.52-3.60 (m, 1H), 3.76 (t, 1H, J=9.2 Hz, 1H), 3.83 (t, J=6.8 Hz, 1H), 3.94-4.20 (m, 6H), 4.30 (dd, J=5.6, 12.8 Hz, 1H), 4.42-4.52 (m, 3H), 4.85, 4.87 (2d, J=8.0 Hz, 1H), 4.92 (dd, J=3.2, 10.4 Hz, 1H), 5.06, 5.08 (2d, J=8.0 Hz, 1H), 5.15 (t, J=9.2 Hz, 1H), 5.30 (d, J=2.8 Hz, 1H), 5.56-5.78 (m, 2H). ¹³C NMR (100 MHz, CDCl₃): δ 20.2, 20.3, 20.4, 20.5, 20.6, 20.7, 20.8, 27.9, 60.8, 61.9, 64.8, 66.5, 66.5, 69.1, 70.6, 71.0, 71.6, 72.7, 72.8, 76.2, 76.7, 99.3, 101.1, 127.8, 130.3, 169.1, 169.6, 169.8, 170.1, 170.18, 170.3, 170.4.

### 4.19 Synthesis of 3-(perfluorooctyl)propanyloxybutanyl-4-O-β-D-galactopyranosyl)-β-D-glucopyranoside (10)

To a solution of 3-(perfluorooctyl)propanyloxybutenyl-4-O-(2,3,4,6-tetra-O-acetyl)-β-D-galactopyranosyl)-2,3,6-tri-O-acetyl-β-D-glucopyranoside (25) (0.1 g, 0.1 mmol) in methanol (5 mL) was added 5% Pd/C (250 mg). The reaction mixture was stirred at ambient temperature under hydrogen atmosphere for 2 h. The reaction mixture was then filtered over Celite and partially concentrated under reduced pressure. To this solution, K₂CO₃ (66 mg) was added and the mixture was stirred at ambient temperature for 3 h. The reaction mixture was neutralized with Amberlyst-15 ion-exchange resin and filtered over Celite. The solvent was removed under reduced pressure to obtain 10 (75 mg, 99%) as a solid.

¹H NMR (400 MHz, CDCl₃): δ 1.50-1.70 (m, 4H), 1.80-1.90 (m, 2H), 2.10-2.40 (m, 2H), 3.10-4.00 (m, 20H), 4.26 (d, J=8.0 Hz, 1H), 4.33 (d, J=7.2 Hz, 1H). ¹³C NMR (100 MHz, CDCl₃): δ 25.9, 26.1, 27.4, 60.6, 61.1, 68.6, 68.9, 69.2, 70.3, 71.1, 73.4, 73.5, 75.0, 75.1, 75.7, 102.8, 103.7.

### 4.20 Synthesis of 3-perfluorooctyl)propanyloxybutenyl-4-O-(2,3,4,6-tetra-O-acetyl-α-D-glucopyranosyl)-2,3,6-tri-O-acetyl-β-D-glucopyranoside (26)

To a solution of 4-O-(2,3,4,6-tetra-O-acetyl-x-D-glucopyranosyl)-2,3,6-tri-O-acetyl-α-D-glucopyranosyl trichloroacetimidate (20) (0.16 g, 0.18 mmol) and 3-(perfluorooctyl)propanyloxybutenyl alcohol (3)(1.0 g, 1.8 mmol) in dichloromethane (20 mL) was added TMSOTf (0.13 mL, 0.15 mmol) at −15° C. The reaction mixture was stirred at −15° C. for 30 min. The reaction was quenched with triethylamine (0.2 mL) and concentrated under reduced pressure. The crude product was purified by solid-phase extraction using a fluorous solid-phase extraction cartridge. Nonfluorous compounds were eluted with 80% MeOH/water and the desired product was eluted by 100% MeOH. The solvent was removed under reduced pressure to obtain 26 (0.12 g, 54%) as a solid.

¹H NMR (400 MHz, CDCl₃): δ 1.80-1.90 (m, 2H), 1.96, 1.97, 1.98, 1.99, 2.00, 2.06, 2.10 (7s, 21H), 1.94-2.40 (m, 2H), 3.45 (t, J=6.0 Hz, 2H), 3.60-3.70 (m, 1H), 3.84-34.08 (m, 5H), 4.10-4.26 (m, 3H), 4.30 (dd, J=5.6, 12.8 Hz, 1H), 4.46 (dd, J=2.4, 12.0 Hz, 1H), 4.52 (d, J=8.0 Hz, 1H), 4.74-4.86 (m, 2H), 5.02 (t, J=10.0 Hz, 1H), 5.20 (t, J=9.2 Hz, 1H), 5.38 (d, J=4.0 Hz, 1H), 5.40-5.80 (m, 2H). ¹³C NMR (100 MHz, CDCl₃): δ 20.2, 20.3, 20.4, 20.5, 20.6, 20.7, 20.8, 20.9, 27.7, 27.9, 61.5, 62.7, 64.7, 66.5, 68.0, 68.5, 68.8, 69.3, 70.0, 72.0, 72.1, 72.2, 72.6, 75.4, 76.7, 95.5, 99.0, 127.6, 130.4, 169.4, 169.6, 169.9, 170.2, 170.4, 170.5.

### 4.21 Synthesis of 3-(perfluorooctyl)propanyloxybutanyl-4-O-β-D-glucopyranosyl)-β-D-glucopyranoside (11)

To a solution of 3-(perfluorooctyl)propanyloxybutenyl-4-O-(2,3,4,6-tetra-O-acetyl)-α-D-glucopyranosyl)-2,3,6-tri-O-acetyl-β-D-glucopyranoside (26) (0.12 g, 0.1 mmol) in methanol (5 mL) was added 5% Pd/C (0.25 g). The reaction mixture was stirred at ambient temperature under hydrogen atmosphere for 2 h. The reaction mixture was then filtered over Celite and partially concentrated under reduced pressure. To this solution, K₂CO₃ (66 mg) was added and the mixture was stirred at ambient temperature for 3 h. The reaction mixture was neutralized with Amberlyst-15-ion-exchange resin and filtered over Celite. The solvent was removed under reduced pressure to obtain 11 (90 mg, 98%) as a solid.

¹H NMR (400 MHz, CDCl₃): δ 1.52-1.70 (m, 4H), 1.74-1.90 (m, 2H), 2.10-2.40 (m, 2H), 3.10-3.38 (m, 4H), 3.40-3.70 (m, 121-1), 3.72-4.00 (m, 4H), 4.24 (d, J=8.0 Hz, 1H), 5.12 (d, J=3.2 Hz, 1H). ¹³C NMR (100 MHz, CDCl₃): δ 20.4, 25.9, 26.1, 27.4, 27.6, 60.8, 61.3, 68.6, 69.2, 70.1, 70.3, 72.7, 73.3, 73.4, 75.2, 76.4, 79.9, 101.5, 102.9.

For the above-stated reasons, it is submitted that the present invention accomplishes at least all of its stated objectives.

Having described the invention with reference to particular compositions and methods, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended.

What is claimed is:

1. A microarray for detecting fluorous-tagged probes comprising:
a substrate having a fluorous surface capable of non-covalently attaching fluorous tagged probes.

2. The microarray of claim 1 whereby the fluorous tagged probe is selected from the group consisting of proteins, fragments of proteins, nucleotides, carbohydrates, molecular complexes, reactive complexes, lipids, fatty acids, steroids, drugs, cells, tissue, and combinations of the same.

3. The microarray of claim 1 whereby the substrate is selected from the group consisting of silica, glass, metal, plastic, and polymers.

4. The microarray of claim 1 whereby the substrate is coated with Teflon.

5. The microarray of claim 1 whereby the substrate is selected from the group consisting of a slide, plate, and well.

6. The microarray of claim 1 further including at least one fluorous tagged probe.

7. The microarray of claim 6 whereby the probe is tagged with a compound selected from the group consisting of fluorous levulinate and a fluorous allyl linker.

8. A method of forming a microarray comprising:
reacting a compound having a fluorous group on the surface of a substrate to form a fluorous surface on the plate or the slide; and
attaching fluorous tagged probes to the fluorous surface.

9. The method of claim 8 whereby the substrate is immersed in a fluorous solution.

10. The method of claim 9 whereby the fluorous solution is an Rf8-reagent.

11. The method of claim 10 whereby the Rf8-reagent is Rf8-ethyl-SiCl₃.

12. A method of detecting a target material, comprising:
preparing a substrate having a fluorous surface;
immobilizing fluorous-tagged probes in the spot regions to prepare a microarray;
contacting the probes and a sample containing the target material to react the probes and the target material; and
detecting the reaction between the target material and the probes.

13. The method of claim 12 whereby the probes are selected from the group consisting of proteins, fragments of proteins, nucleotides, carbohydrates, molecular complexes, reactive complexes, lipids, fatty acids, steroids, drugs, cells, tissue, and combinations of the same.

14. A compound used to facilitate synthesis and arraying of compounds comprising fluorous levulinate.

15. A compound used to facilitate synthesis and arraying of compounds comprising a fluorous allyl linker.
